(19) United States
(12) Patent Application Publication
(10) Pub. No.: US 2021/0346596 A1
(43) Pub. Date: Nov. 11, 2021

(54) PLATFORM ASSEMBLY PROCESS FOR DRUG DELIVERY DEVICE

(71) Applicant: AMGEN INC., Thousand Oaks, CA (US)

(72) Inventors: Jakob Halkjaer Pedersen, Frederiksberg (DK); Matias Melander, Copenhagen (DK); Christian Plambech, Soeborg (DK); Adam B. McCullough, Westlake Village, CA (US); Rasmus Ohlenschlager, Gentofte (DK)

(21) Appl. No.: 17/277,533

(22) PCT Filed: Oct. 15, 2019
(86) PCT No.: PCT/US2019/056175

§ 371 Date: Mar. 18, 2021

Related U.S. Application Data
(60) Provisional application No. 62/745,739, filed on Oct. 15, 2018.

(51) Int. Cl. A61M 5/14; A61M 5/24; A61M 5/20

(52) U.S. Cl. CPC A61M 5/1413; A61M 5/24; A61M 2205/02; A61M 2207/00; A61M 5/2033

(57) ABSTRACT

An approach for assembling a platform drug delivery device includes providing a set of base components and identifying, based on at least one desired characteristic of the platform drug delivery device, a rear sub-assembly for the drug delivery device from a group of rear sub-assemblies. The identified rear sub-assembly is selected, and a front sub-assembly is identified based on the at least one desired characteristic from a group of front sub-assemblies. The identified front-assembly is selected, and the drug delivery device is assembled using the set of base components, the rear sub-assembly, and the front sub-assembly.

### CROSS-REFERENCE TO RELATED APPLICATION

Priority is claimed to U.S. Provisional Patent Application No. 62/745,739, filed Oct. 15, 2018, the entirety of which is hereby incorporated herein by reference.

### FIELD OF DISCLOSURE

The present disclosure generally relates to drug delivery devices and, more particularly, to platform manufacturing approaches for drug delivery devices.

### BACKGROUND

Drug delivery devices such as autoinjectors and on-body injectors offer several benefits in delivery of medicaments and/or therapeutics. One of the benefits can include simplicity of use, as compared with traditional methods of delivery using, for example, conventional syringes.

Autoinjectors may be used to deliver a number of different drugs having varying viscosities and/or desired volumes. As a result, assembly of these devices can be complex due to the need to properly identify suitable components that can effectively deliver the medicament to the user. As an example, drugs having higher viscosities may require stronger drive assemblies having more robust components to adequately deliver the drug within reasonable time frames. Similarly, larger doses of drugs may also require more robust drive assemblies.

### SUMMARY

In accordance with a first aspect, an approach for assembling a platform drug delivery device includes providing a set of base components and identifying, based on at least one desired characteristic of the platform drug delivery device, a rear sub-assembly for the drug delivery device from a group of rear sub-assemblies. The identified rear sub-assembly is selected, and a front sub-assembly is identified based on the at least one desired characteristic from a group of front sub-assemblies. The identified front-assembly is selected, and the drug delivery device is assembled using the set of base components, the rear sub-assembly, and the front sub-assembly. The approach may optionally include applying a skin to the device, which may be selected based on at least one attribute from an intended user group.

In some aspects, the at least one desired characteristic is in the form of at least one of a drug viscosity or a drug volume. In some aspects, each of the rear sub-assemblies in the group of rear sub-assemblies may include a different drive mechanism. Further, each of the front sub-assemblies may include a different syringe assembly, which may be constructed from one of glass or a polymeric material. In some examples, the set of base components are geometrically identical between configurations of the drug delivery device.

In accordance with another aspect, an approach of assembling a platform drug delivery device includes providing a set of base components for the device, identifying a first sub-assembly for the device from a first group of selectable sub-assemblies, and selecting the identified first sub-assembly. A second sub-assembly is identified from a second group of selectable sub-assemblies, and the second sub-assembly is selected. A third sub-assembly is also identified from a third group of selectable sub assemblies, and the third sub-assembly is selected. The drug delivery device is assembled using the set of base components, the first sub-assembly, the second sub-assembly, and the third sub-assembly.

In accordance with a third aspect, a platform drug delivery device is prepared by a process that includes the steps of providing a set of base components for the device, identifying a first sub-assembly for the device from a first group of selectable sub-assemblies, and selecting the identified first sub-assembly. A second sub-assembly is identified from a second group of selectable sub-assemblies, and the second sub-assembly is selected. A third sub-assembly is also identified from a third group of selectable sub assemblies, and the third sub-assembly is selected. The drug delivery device is assembled using the set of base components, the first sub-assembly, the second sub-assembly, and the third sub-assembly.

In accordance with a fourth aspect, a platform system for a drug delivery device includes a set of base components for the drug delivery device, a first group of selectable sub-assemblies for the drug delivery device, a second group of selectable sub-assemblies for the drug delivery device, and a third group of selectable sub-assemblies for the drug delivery device. The drug delivery device is assembled by using at least one desired characteristic of the drug delivery device to identify and select a first sub-assembly from the first group of selectable sub-assemblies, a second sub-assembly from the second group of selectable sub-assemblies, and a third sub-assembly from the third group of selectable sub-assemblies. The set of base components is coupled to the first group of selectable sub-assemblies, the second group of selectable sub-assemblies, and the third group of selectable sub-assemblies.

### BRIEF DESCRIPTION OF THE DRAWINGS

The above needs are at least partially met through provision of the platform assembly process for a delivery device described in the following detailed description, particularly when studied in conjunction with the drawings, wherein:

FIG. 1 illustrates an example approach to assembling a platform drug delivery device in accordance with various embodiments;

FIG. 2 illustrates an example approach for supply chain and assembly of a platform drug delivery device in accordance with various embodiments;

FIG. 3 illustrates an example first approach for applying a skin to a drug delivery device in accordance with various embodiments;

FIG. 4 illustrates a second approach for applying a skin to a drug delivery device in accordance with various embodiments

FIG. 5 illustrates example pre-filled syringes having different material characteristics for use with a platform drug delivery device in accordance with various embodiments;

FIG. 6 illustrates a zoomed-in view of the example pre-filled syringes of FIG. 5 in accordance with various embodiments;

FIG. 7 illustrates the example pre-filled syringes of FIGS. 5 and 6 being installed in a drug delivery device in accordance with various embodiments;

FIGS. 8a and 8b illustrate a first example pre-filled syringe having a first example support structure in accordance with different embodiments;

FIGS. 9a and 9b illustrate a second example pre-filled syringe having a second example support structure in accordance with different embodiments; and

FIGS. 10a and 10b illustrate a third example pre-filled syringe having a third example support structure in accordance with different embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, a drug delivery device can include a housing, a syringe assembly containing a medicament to be injected into a user, and an actuating assembly that includes a drive mechanism (e.g., a torque spring) to cause the medicament to be injected into the user. As the drive mechanism rotates to cause the drug to be administered, different forces may be required to efficiently and completely deliver the drug to the user. An example drug delivery device is described in U.S. App. No. 62/719,367 filed on Sep. 17, 2018, the contents of which are herein incorporated by reference in its entirety. The approaches described herein cover a large range of drug fluid volumes and viscosities, and allows for further customization for user groups. Additionally, the approaches described herein enable a number of components to be reused across drug products, thereby enabling investment in higher cavity tooling to reduce costs. As a result, drug delivery devices may be more likely to be ready for required clinical trials shortly after the process for determining the appropriate drug dosage (e.g., an appropriate volume and concentration) has occurred.

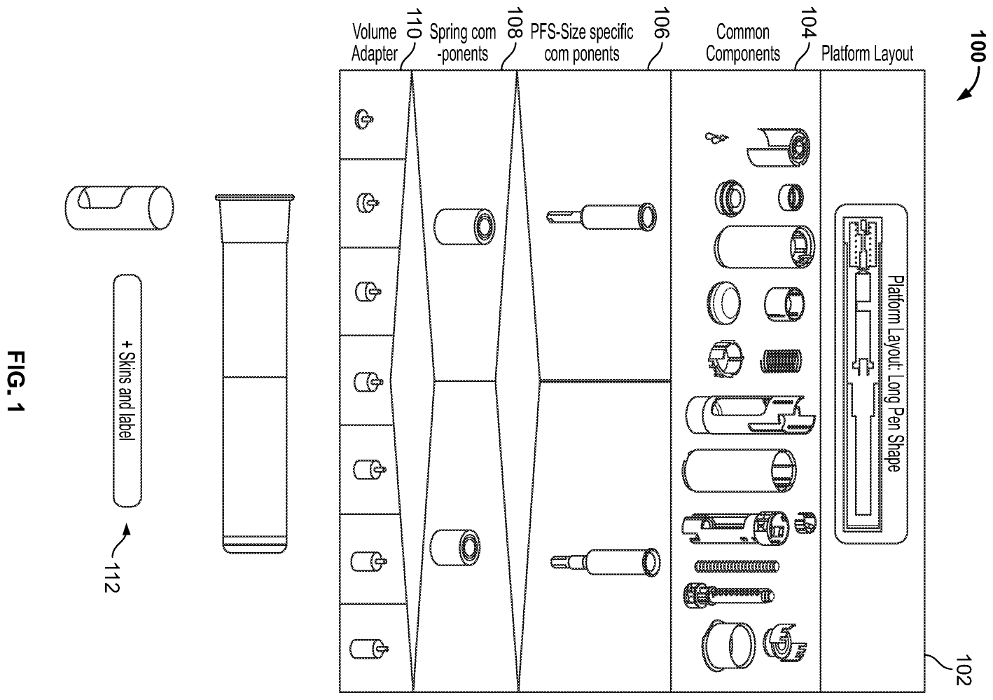

With reference to FIG. 1, an assembly approach for a platform drug delivery device 100 considers any number of base configurations as devices that support varying sub-assemblies which may be used to address different technical requirements (e.g., dosing times for combinations of volumes and/or drug viscosities). Additionally, any number of “adaptions” may be used to cater to different user groups, markets, and the like to create a better user experience and/or market differentiation.

The different configurations may reuse any number of components, but can differ in a select number of areas to provide the desired output device. At a first or top level 102, an example platform layout is illustrated which provides information pertaining to the basic device layout. While the example top level 102 illustrates a “long pen shape” device, any number of desired devices may be illustrated at the top level 102.

At the next level, a set of base or common components 104 are provided that are geometrically identical between all possible configurations of the top level device. These components can include, but are not limited to, a housing, a shield member, a spring housing, a syringe holder, a plunger rod, a plunger rod guide, a cap, a nut, a shield spring, an end of dosage clicking device, a trigger ring, a shield lock, a top housing, a damper member, a spring guide, and/or damper grease. Other base components 104 may also be provided.

The remaining components and/or sub-assemblies may be identified and selected based on at least one desired characteristic of the drug delivery device. For example, a desired drug having a specified viscosity and/or viscosity range, a specified volume, etc. may be used to identify these components. A first sub-assembly 106 may include pre-filled syringe (“PFS”) shape specific components. These components may include a syringe barrel, a portion of the needle assembly, and the like. In the illustrated platform 100, the first sub-assembly 106 only includes two options which only differ in the PFS used due to optimization of both PFSs and their supporting components, but it is understood that the first sub-assembly may include any number of distinct assemblies having any number of individual components therein.

A second sub-assembly 108 may include spring components. This level illustrates how many different drive mechanisms are being used in a platform in order to support the various drug fluid volumes and/or viscosities within requirements for dosing times. In this example, the illustrated drive mechanisms only differ in height, and not length, thickness, or other processing. Importantly, the base components (e.g., the plunger rod guide, the housing, and the spring guide) have been designed to accept the variance in drive mechanism dimensions. While the illustrated sub-assembly 108 only includes two options, it is understood that the second sub-assembly may include any number of distinct assemblies having any number of components therein.

A third sub-assembly 110 may include volume adapters. This level illustrates how the platform configurations may be adapted to work optimally with different drug fluid volumes. For example, some drug delivery devices may include dampers that are used in part to reduce impact seed on lower PFS fill volumes. Accordingly, some components (e.g., the plunger rod) may take a longer time to move down to the plunger in the PFS. A volume adapter may be used to occupy some of this space and thereby reduce the overall injection time for low volume configurations. It will be appreciated that the third sub-assembly may also include any number of distinct assemblies having any number of components therein.

As configured, the platform assembly described herein may be used with drugs having viscosities between approximately 1 cP and approximately 30 cP, and deliverable volumes between approximately 0.2 ml and approximately 3.5 ml. However, in other examples, drugs having increased or decreased viscosities as well as varying volumes of drugs may be used.

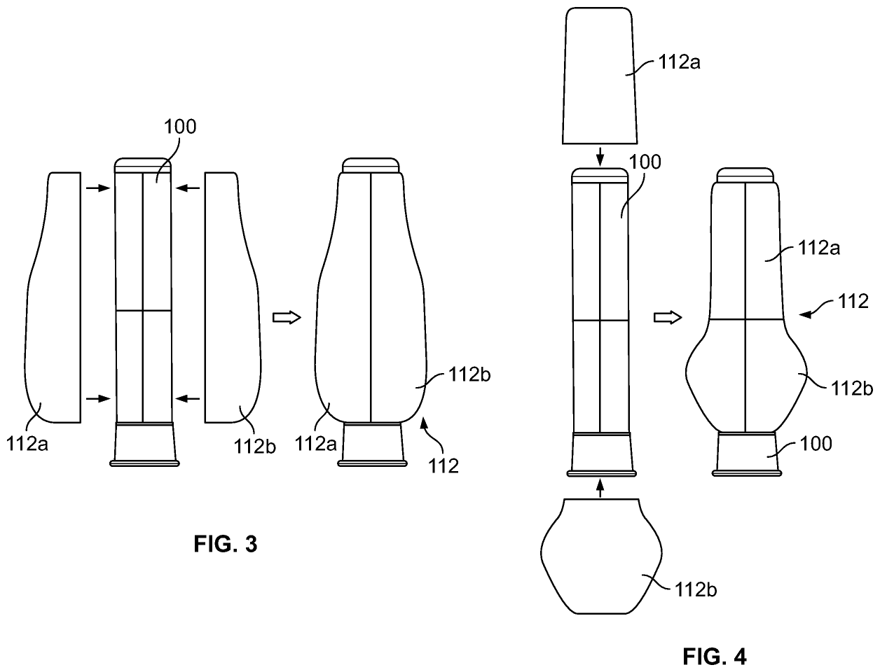

The platform device 100 may also include a skin level 112 to allow the device to be adapted to different user populations and/or markets. For example, as illustrated in FIGS. 3 and 4, the skins 112 are provided in the form of a shell having two portions 112a, 112b to the outside of the device 100. As illustrated in FIG. 3, the shell 112 includes two sides or halves that are longitudinally aligned, and in FIG. 4, the shell 112 includes a top portion and a bottom portion that couple together near a midpoint of the device 100. Other examples are possible.

In some approaches, the skins 112 may also be selected based on a desired attribute. For example, the skin 112 may be selected based on an attribute from an intended user group, such as, for example, whether the drug is administered by a healthcare professional, whether the device is intended for individuals with certain limiting ailments (e.g., rheumatoid arthritis, migraines, etc.) that may need ergonomic affordances such as larger or smaller grip portions, and the like.

In the platform assembly process, it is of particular importance to manufacture different configurations as efficiently as possible. One approach to accomplishing this efficiency is by pushing the variant creation to occur at a late stage in the assembly process, such as by providing varying rear sub-assemblies (“RSA”) and front sub-assemblies (“FSA”). In these examples, the provided RSAs only differ in the particular drive mechanism (e.g., watch spring) being implemented. These sub-assemblies are typically stored unwound to minimize the risk of creep or disassembly during storage from the high forces being contained inside the module and to allow a varying amount of load in the wound spring depending on the drug used. As previously noted, the particular spring used depends on the desired drug volume and/or viscosity of the desired drug. In one example, the variation in spring size is used to accommodate injection times between approximately four seconds and approximately 10 seconds for a particular drug volume/viscosity relationship.

In the provided example, the FSA can accommodate any number (e.g., two or more) PFS designs constructed from any number of different materials (e.g., glass, a polymeric material such as cyclic olefin copolymer or cyclic olefin polymer, etc.). This variation advantageously accommodates different drug products that may not be compatible with certain components (e.g., silicone oil, which may be a requirement for glass syringes). Similarly, low viscosity products may incur flow issues across components of polymeric PFS devices. Accordingly, by accommodating these requirements provides a high likelihood of being suitable for a large number of drug products. In some examples, by customizing the syringe holder component to be a generic device that has interfaces for multiple types of PFS devices (e.g., syringes constructed from glass or plastic materials) allows for a single sub-assembly design that only differs by the particular PFS being used. In these examples, minimal changes may be required to the FSA to accommodate different PFS designs. For example, a needle shield to cap interface may be modified as needed.

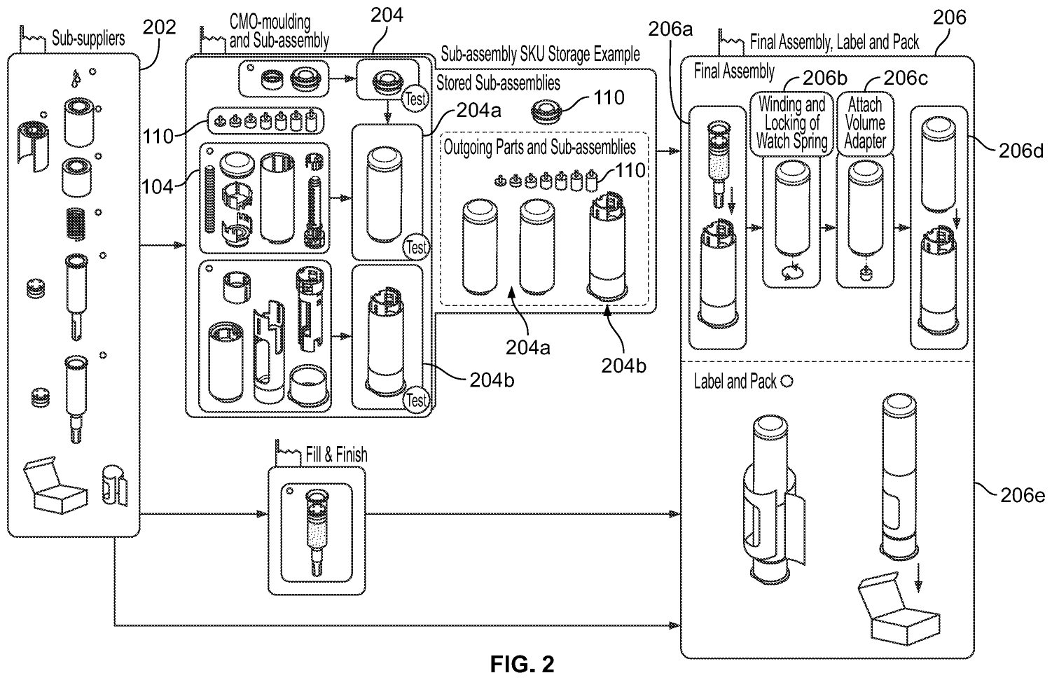

Turning now to FIG. 2, an example approach for supply chain and assembly of a platform drug delivery device is provided. In some of these examples, the winding of the drive mechanism may be adjusted during final assembly. As a result, adjustable feedback mechanisms may be used that uses the number of winding turns to identify how far, or how many turns, must be made before dose completion. Such a feature may be useful in end-of-dose indicator applications. It is appreciated that while not described in substantial detail herein, any number of skins may be applied to the device during final assembly stages along with a device label.

At a first level 202, components required to assemble the devices are supplied and stocked by sub-suppliers. At a second level 204, sub-assemblies are prepared and stored as different SKUs. As illustrated in box 204, two RSAs 204a are assembled having differing drive mechanisms, and a single FSA 204b is assembled. The overall stored sub-assemblies can include the RSAs, FSAs, and any additional common components such as dampers.

Next, the desired characteristics for the device are identified. For example, a particular drug may be desired having a particular required dose volume. As illustrated in box 206, during the final assembly, labeling, and packing phase, at a step 206a, the first sub-assembly 106 is assembled by inserting the desired PFS into the FSA. The PFS may be filled at any time during the assembly process using any number of approaches. In some examples, the PFS may be filled at different locations (e.g., separate facilities having clean environments) and may subsequently be shipped to a final assembly site. Further, at a step 206b, the desired second sub-assembly is assembled, and the drive mechanism is inserted into the RSA. If the device incorporates a third sub-assembly in the form of a volume adapter, it is attached at a step 206c. Next, the FSA is coupled to the RSA at a step 206d. Finally, the device is labeled and packaged at a step 206e.

Typically, PFSs come in standard configurations with container volumes and sizes governed by international standards. Accordingly, PFSs are generally dimensionally the same across suppliers. By using the platform approaches described herein, PFS designs may be optimized for improved robustness in the interface with the device. Typically, a PFS is supported by the flange when mounted in an auto-injector. Since especially glass PFSs can have large tolerances on the length of the syringe barrel, the tolerance stack-ups for the PFS, and auto-injector combination often lead to large variability in needle extension and plunger position relatively to the auto-injector components. Supporting the PFS by the shoulder can reduce this variability.

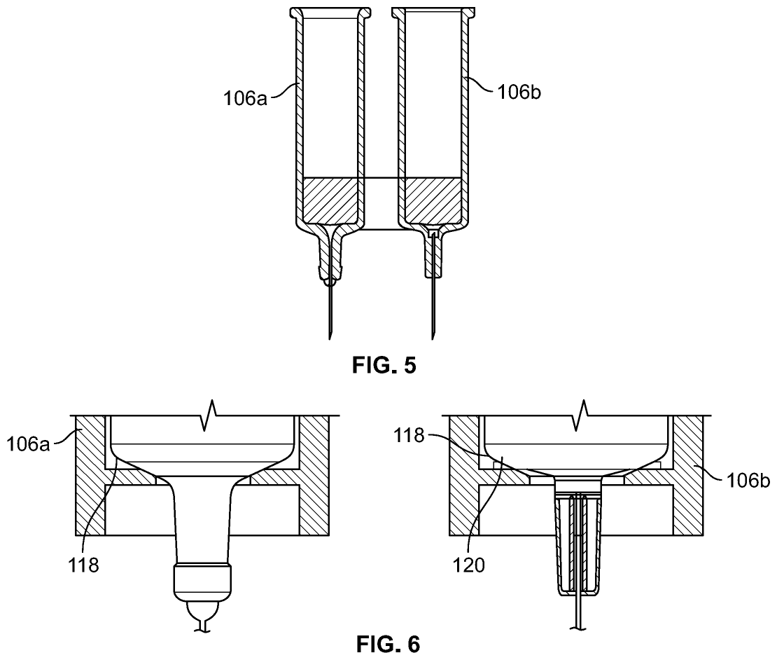

FIG. 5 illustrates two example PFS assemblies 106 for use with the platform device 100. The PFS 106a is constructed from a glass material, and the PFS 106b is constructed from a polymeric material such as COP. In the PFSs 106a, 106b, the height of the two plunger-stoppers is optimized to allow for the same plunger back position at the end of dose, which advantageously assists in supporting potential end-of-dose feedback functions in the device. Specifically, in some applications, a mechanical trigger may provide end-of-dose feedback in varying forms to signal to the user that the dose delivery is complete. This trigger needs to occur while components are still moving in order to enable the trigger, but needs to occur as close as possible to the actual end of dosing (i.e., when the plunger rod and the stopper bottom out within the PFS). The plunger rod (or other component directly coupled to the plunger rod) may be used to implement the feedback functionality, though importantly, if the stroke of the plunger rod is terminated at a different location based on differing container and/or stopper dimensions, the feedback trigger mechanism would need to be adjusted between the platform variants. However, the presently-described platform design includes a FSA and PFS that avoid different end of dose termination positions of the plunger so that no additional application-specific components are needed to implement end-of dose feedback for use with the selectable components.

Additionally, these designs are optimized while accounting for minimum distance requirements between the sealing ribs and the diameter-to-height ratio of the plunger to enable orientation during feeding in vibrator bowls. Further, the outer diameters of the PFSs 106a, 106b are identical or near-identical to avoid the need for device-specific parts for each PFS. The dimensions of the PFS can be divided into two groups: interface and non-interface dimensions. Example interface dimensions include the overall length and diameter of the PFSs 106a, 106b. As noted, the example PFSs 106a, 106b have the same overall length (e.g., the length from the needle tip to the PFS support and/or the flange back to the PFS support) and diameters. Example non-interface dimensions include similar flange heights and diameters as well as inner diameters.

With reference to FIGS. 5, 6, and 8a-10b, in some examples, the PFS 106b is constructed via injection molding approaches which provide for additional freedom in terms of feature design when compared to the glass PFS 106a. The PFS 106b design advantageously includes a support feature 120 disposed on a shoulder 118 of the syringe barrel. The support 120 provides for a less ambiguous interface to the device that is easily controllable. For example, with reference to FIGS. 8a-10b, three example PFSs 106a constructed from a polymeric material are provided. As shown in FIGS. 8a and 8b, the PFS 106a includes a support feature 120 in the form of a plurality of ribs extending radially from the shoulder surface 118. As shown in FIGS. 9a and 9b, the PFS 106a includes a support feature 120 in the form of a surface or protrusion extending outwardly from the shoulder surface 118, and as shown in FIGS. 10a and 10b, the PFS 106a includes a support feature 120 in the form of a ring protruding from the shoulder surface 118. Other examples are possible.

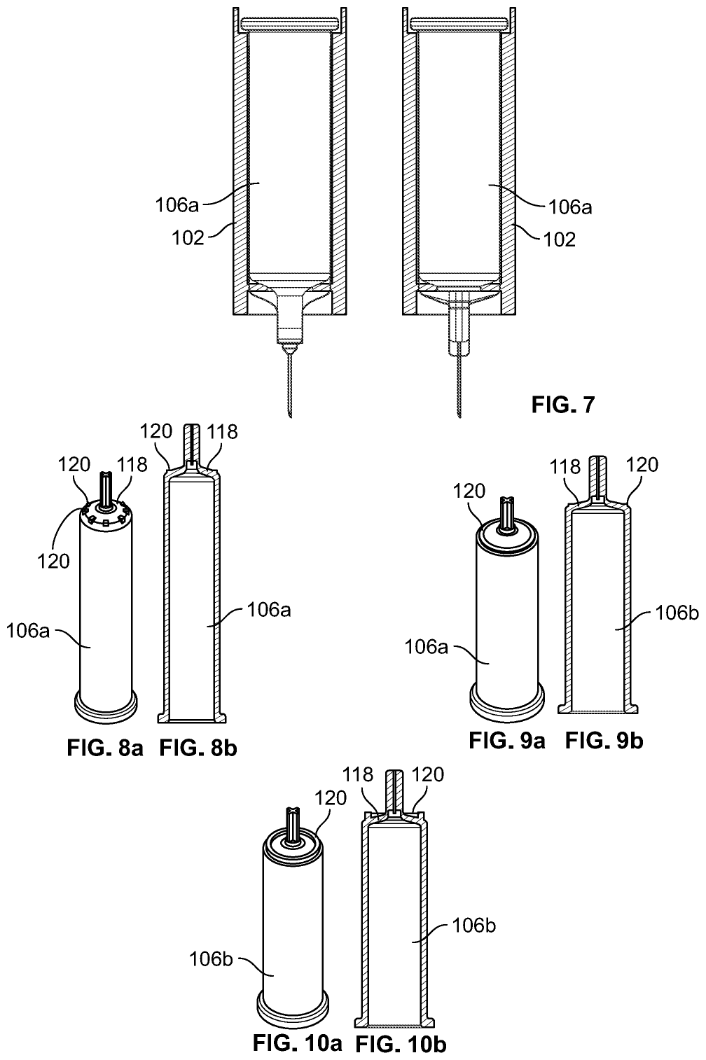

Additionally, the interface between the outer surface of the PFS and the inner diameter of the device may be advantageously designed. The interface may be in the form of full cylinder contact throughout the barrel length (as illustrated in FIG. 7), ribs on or along the length of the barrel (not shown), rings around the circumference of the barrel (not shown), and/or small protrusions or dots positioned on the surface of the device or the barrel (not shown). So configured, a syringe carrier component may be provided that includes dual support surfaces that are compatible with both glass and plastic PFS devices.

Advantageously, the described platform approach eliminates the need for numerous final device stock keeping units (“SKUs”) for each drug product to be used in the autoinjectors. In order to properly manage a supply chain and inventory would otherwise require an identical number of sub-assemblies that each need a minimum inventory level based on anticipated product demand. However, the present platform approach utilizes a single front sub-assembly to serve the needs of all of the various drug products, and a combination of additional rear sub-assemblies will also serve all of these products. Such flexibility to apply the same sub-assemblies to different drug products provides a more nimble supply chain that in turn reduces the total value of inventory maintained without increasing the risk of backorder.

The above description describes various assemblies, devices, and methods for use with a drug delivery device. It should be clear that the assemblies, drug delivery devices, or methods can further comprise use of a medicament listed below with the caveat that the following list should neither be considered to be all inclusive nor limiting. The medicament will be contained in a reservoir. In some instances, the reservoir is a primary container that is either filled or pre-filled for treatment with the medicament. The primary container can be a cartridge or a pre-filled syringe.

For example, the drug delivery device or more specifically the reservoir of the device may be filled with colony stimulating factors, such as granulocyte colony-stimulating factor (G-CSF). Such G-CSF agents include, but are not limited to, Neupogen® (filgrastim) and Neulasta® (pegfilgrastim). In various other embodiments, the drug delivery device may be used with various pharmaceutical products, such as an erythropoiesis stimulating agent (ESA), which may be in a liquid or a lyophilized form. An ESA is any molecule that stimulates erythropoiesis, such as Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, as well as the molecules or variants or analogs thereof as disclosed in the following patents or patent applications, each of which is herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,986,047; 6,583,272; 7,084,245; and 7,271,689; and PCT Publication Nos. WO 91/05867; WO 95/05465; WO 96/40772; WO 00/24893; WO 01/81405; and WO 2007/136752.

An ESA can be an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, epoetin alfa, epoetin beta, epoetin delta, epoetin omega, epoetin iota, epoetin zeta, and analogs thereof, pegylated erythropoietin, carbamoylated erythropoietin, mimetic peptides (including EMP1/hematide), and mimetic antibodies. Exemplary erythropoiesis stimulating proteins include erythropoietin, darbepoetin, erythropoietin agonist variants, and peptides or antibodies that bind and activate erythropoietin receptor (and include compounds reported in U.S. Publication Nos. 2003/0215444 and 2006/0040858, the disclosures of each of which is incorporated herein by reference in its entirety) as well as erythropoietin molecules or variants or analogs thereof as disclosed in the following patents or patent applications, which are each herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,830,851; 5,856,298; 5,986,047; 6,030,086; 6,310,078; 6,391,633; 6,583,272; 6,586,398; 6,900,292; 6,750,369; 7,030,226; 7,084,245; and 7,217,689; U.S. Publication Nos. 2002/0155998; 2003/0077753; 2003/0082749; 2003/0143202; 2004/0009902; 2004/0071694; 2004/0091961; 2004/0143857; 2004/0157293; 2004/0175379; 2004/0175824; 2004/0229318; 2004/0248815; 2004/0266690; 2005/0019914; 2005/0026834; 2005/0096461; 2005/0107297; 2005/0107591; 2005/0124045; 2005/0124564; 2005/0137329; 2005/0142642; 2005/0143292; 2005/0153879; 2005/0158822; 2005/0158832; 2005/0170457; 2005/0181359; 2005/0181482; 2005/0192211; 2005/0202538; 2005/0227289; 2005/0244409; 2006/0088906; and 2006/0111279; and PCT Publication Nos. WO 91/05867; WO 95/05465; WO 99/66054; WO 00/24893; WO 01/81405; WO 00/61637; WO 01/36489; WO 02/014356; WO 02/19963; WO 02/20034; WO 02/49673; WO 02/085940; WO 03/029291; WO 2003/055526; WO 2003/084477; WO 2003/094858; WO 2004/002417; WO 2004/002424; WO 2004/009627; WO 2004/024761; WO 2004/033651; WO 2004/035603; WO 2004/043382; WO 2004/101600; WO 2004/101606; WO 2004/101611; WO 2004/106373; WO 2004/018667; WO 2005/001025; WO 2005/001136; WO 2005/021579; WO 2005/025606; WO 2005/032460; WO 2005/051327; WO 2005/063808; WO 2005/063809; WO 2005/070451; WO 2005/081687; WO 2005/084711; WO 2005/103076; WO 2005/100403; WO 2005/092369; WO 2006/50959; WO 2006/02646; and WO 2006/29094.

Examples of other pharmaceutical products for use with the device may include, but are not limited to, antibodies such as Vectibix® (panitumumab), Xgeva™ (denosumab) and Prolia™ (denosamab); other biological agents such as Enbrel® (etanercept, TNF-receptor/Fc fusion protein, TNF blocker), Neulasta® (pegfilgrastim, pegylated filgastrim, pegylated G-CSF, pegylated hu-Met-G-CSF), Neupogen® (filgrastim, G-CSF, hu-MetG-CSF), and Nplate® (romiplostim); small molecule drugs such as Sensipar® (cinacalcet). The device may also be used with a therapeutic antibody, a polypeptide, a protein or other chemical, such as an iron, for example, ferumoxytol, iron dextrans, ferric glyconate, and iron sucrose. The pharmaceutical product may be in liquid form, or reconstituted from lyophilized form.

Among particular illustrative proteins are the specific proteins set forth below, including fusions, fragments, analogs, variants or derivatives thereof:

OPGL specific antibodies, peptibodies, and related proteins, and the like (also referred to as RANKL specific antibodies, peptibodies and the like), including fully humanized and human OPGL specific antibodies, particularly fully humanized monoclonal antibodies, including but not limited to the antibodies described in PCT Publication No. WO 03/002713, which is incorporated herein in its entirety as to OPGL specific antibodies and antibody related proteins, particularly those having the sequences set forth therein, particularly, but not limited to, those denoted therein: 9H7; 18B2; 2D8; 2E11; 16E1; and 22B3, including the OPGL specific antibodies having either the light chain of sequence identification number:2 as set forth therein in FIG. 2 and/or the heavy chain of sequence identification number:4, as set forth therein in FIG. 4, each of which is individually and specifically incorporated by reference herein in its entirety fully as disclosed in the foregoing publication;

Myostatin binding proteins, peptibodies, and related proteins, and the like, including myostatin specific peptibodies, particularly those described in U.S. Publication No. 2004/0181033 and PCT Publication No. WO 2004/058988, which are incorporated by reference herein in their entirety particularly in parts pertinent to myostatin specific peptibodies, including but not limited to peptibodies of the mTN8-19 family, including those of sequence identification numbers:305-351, including TN8-19-1 through TN8-19-40, TN8-19 con1 and TN8-19 con2; peptibodies of the mL2 family of sequence identification numbers:357-383; the mL15 family of sequence identification numbers:384-409; the mL17 family of sequence identification numbers:410-438; the mL20 family of sequence identification numbers:439-446; the mL21 family of sequence identification numbers:447-452; the mL24 family of sequence identification numbers:453-454; and those of sequence identification numbers:615-631, each of which is individually and specifically incorporated by reference herein in their entirety fully as disclosed in the foregoing publication;

IL-4 receptor specific antibodies, peptibodies, and related proteins, and the like, particularly those that inhibit activities mediated by binding of IL-4 and/or IL-13 to the receptor, including those described in PCT Publication No. WO 2005/047331 or PCT Application No. PCT/US2004/37242 and in U.S. Publication No. 2005/112694, which are incorporated herein by reference in their entirety particularly in parts pertinent to IL-4 receptor specific antibodies, particularly such antibodies as are described therein, particularly, and without limitation, those designated therein: L1H1; L1H2; L1H3; L1H4; L1H5; L1H6; L1H7; L1H8; L1H9; L1H10; L1H11; L2H1; L2H2; L2H3; L2H4; L2H5; L2H6; L2H7; L2H8; L2H9; L2H10; L2H11; L2H12; L2H13; L2H14; L3H1; L4H1; L5H1; L6H1, each of which is individually and specifically incorporated by reference herein in its entirety fully as disclosed in the foregoing publication;

Interleukin 1-receptor 1 (“IL1-R1”) specific antibodies, peptibodies, and related proteins, and the like, including but not limited to those described in U.S. Publication No. 2004/097712, which is incorporated herein by reference in its entirety in parts pertinent to IL1-R1 specific binding proteins, monoclonal antibodies in particular, especially, without limitation, those designated therein: 15CA, 26F5, 27F2, 24E12, and 10H7, each of which is individually and specifically incorporated by reference herein in its entirety fully as disclosed in the aforementioned publication;

Ang2 specific antibodies, peptibodies, and related proteins, and the like, including but not limited to those described in PCT Publication No. WO 03/057134 and U.S. Publication No. 2003/0229023, each of which is incorporated herein by reference in its entirety particularly in parts pertinent to Ang2 specific antibodies and peptibodies and the like, especially those of sequences described therein and including but not limited to: L1(N); L1(N) WT; L1(N) 1K WT; 2xL1(N); 2xL1(N) WT; Con4 (N), Con4 (N) 1K WT, 2xCon4 (N) 1K; L1C; L1C 1K; 2xL1C; Con4C; Con4C 1K; 2xCon4C 1K; Con4-L1 (N); Con4-L1C; TN-12-9 (N); C17 (N); TN8-8(N); TN8-14 (N); Con 1 (N), also including anti-Ang 2 antibodies and formulations such as those described in PCT Publication No. WO 2003/030833 which is incorporated herein by reference in its entirety as to the same, particularly Ab526; Ab528; Ab531; Ab533; Ab535; Ab536; Ab537; Ab540; Ab543; Ab544; Ab545; Ab546; A551; Ab553; Ab555; Ab558; Ab559; Ab565; AbF1AbFD; AbFE; AbFJ; AbFK; AbG1D4; AbGC1E8; AbH1C12; AblA1; AbIF; AbIK, AbIP; and AbIP, in their various permutations as described therein, each of which is individually and specifically incorporated by reference herein in its entirety fully as disclosed in the foregoing publication;

NGF specific antibodies, peptibodies, and related proteins, and the like including, in particular, but not limited to those described in U.S. Publication No. 2005/0074821 and U.S. Pat. No. 6,919,426, which are incorporated herein by reference in their entirety particularly as to NGF-specific antibodies and related proteins in this regard, including in particular, but not limited to, the NGF-specific antibodies therein designated 4D4, 4G6, 6H9, 7H2, 14D10 and 14D11, each of which is individually and specifically incorporated by reference herein in its entirety fully as disclosed in the foregoing publication;

CD22 specific antibodies, peptibodies, and related proteins, and the like, such as those described in U.S. Pat. No. 5,789,554, which is incorporated herein by reference in its entirety as to CD22 specific antibodies and related proteins, particularly human CD22 specific antibodies, such as but not limited to humanized and fully human antibodies, including but not limited to humanized and fully human monoclonal antibodies, particularly including but not limited to human CD22 specific IgG antibodies, such as, for instance, a dimer of a human-mouse monoclonal hLL2 gamma-chain disulfide linked to a human-mouse monoclonal hLL2 kappa-chain, including, but limited to, for example, the human CD22 specific fully humanized antibody in Epratuzumab, CAS registry number 501423-23-0;

IGF-1 receptor specific antibodies, peptibodies, and related proteins, and the like, such as those described in PCT Publication No. WO 06/069202, which is incorporated herein by reference in its entirety as to IGF-1 receptor specific antibodies and related proteins, including but not limited to the IGF-1 specific antibodies therein designated L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, L52H52, and IGF-1R-binding fragments and derivatives thereof, each of which is individually and specifically incorporated by reference herein in its entirety fully as disclosed in the foregoing publication;

Also among non-limiting examples of anti-IGF-1R antibodies for use in the methods and compositions of the present invention are each and all of those described in:

(i) U.S. Publication No. 2006/0040358 (published Feb. 23, 2006), 2005/0008642 (published Jan. 13, 2005), 2004/0228859 (published Nov. 18, 2004), including but not limited to, for instance, antibody 1A (DSMZ Deposit No. DSM ACC 2586), antibody 8 (DSMZ Deposit No. DSM ACC 2589), antibody 23 (DSMZ Deposit No. DSM ACC 2588) and antibody 18 as described therein;

(ii) PCT Publication No. WO 06/138729 (published Dec. 28, 2006) and WO 05/016970 (published Feb. 24, 2005), and Lu et al. (2004), J. Biol. Chem. 279:2856-2865, including but not limited to antibodies 2F8, A12, and IMC-A12 as described therein;

(iii) PCT Publication No. WO 07/012614 (published Feb. 1, 2007), WO 07/000328 (published Jan. 4, 2007), WO 06/013472 (published Feb. 9, 2006), WO 05/058967 (published Jun. 30, 2005), and WO 03/059951 (published Jul. 24, 2003);

(iv) U.S. Publication No. 2005/0084906 (published Apr. 21, 2005), including but not limited to antibody 7C10, chimaeric antibody C7C10, antibody h7C10, antibody 7H2M, chimaeric antibody *7C10, antibody GM 607, humanized antibody 7C10 version 1, humanized antibody 7C10 version 2, humanized antibody 7C10 version 3, and antibody 7H2HM, as described therein;

(v) U.S. Publication Nos. 2005/0249728 (published Nov. 10, 2005), 2005/0186203 (published Aug. 25, 2005), 2004/0265307 (published Dec. 30, 2004), and 2003/0235582 (published Dec. 25, 2003) and Maloney et al. (2003), Cancer Res. 63:5073-5083, including but not limited to antibody EM164, resurfaced EM164, humanized EM164, huEM164 v1.0, huEM164 v1.1, huEM164 v1.2, and huEM164 v1.3 as described therein;

(vi) U.S. Pat. No. 7,037,498 (issued May 2, 2006), U.S. Publication Nos. 2005/0244408 (published Nov. 30, 2005) and 2004/0086503 (published May 6, 2004), and Cohen, et al. (2005), Clinical Cancer Res. 11:2063-2073, e.g., antibody CP-751,871, including but not limited to each of the antibodies produced by the hybridomas having the ATCC accession numbers PTA-2792, PTA-2788, PTA-2790, PTA-2791, PTA-2789, PTA-2793, and antibodies 2.12.1, 2.13.2, 2.14.3, 3.1.1, 4.9.2, and 4.17.3, as described therein;

(vii) U.S. Publication Nos. 2005/0136063 (published Jun. 23, 2005) and 2004/0018191 (published Jan. 29, 2004), including but not limited to antibody 19D12 and an antibody comprising a heavy chain encoded by a polynucleotide in plasmid 15H12/19D12 HCA (γ4), deposited at the ATCC under number PTA-5214, and a light chain encoded by a polynucleotide in plasmid 15H12/19D12 LCF (K), deposited at the ATCC under number PTA-5220, as described therein; and

(viii) U.S. Publication No. 2004/0202655 (published Oct. 14, 2004), including but not limited to antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, as described therein; each and all of which are herein incorporated by reference in their entireties, particularly as to the aforementioned antibodies, peptibodies, and related proteins and the like that target IGF-1 receptors;

B-7 related protein 1 specific antibodies, peptibodies, related proteins and the like (“B7RP-1,” also is referred to in the literature as B7H2, ICOSL, B7h, and CD275), particularly B7RP-specific fully human monoclonal IgG2 antibodies, particularly fully human IgG2 monoclonal antibody that binds an epitope in the first immunoglobulin-like domain of B7RP-1, especially those that inhibit the interaction of B7RP-1 with its natural receptor, ICOS, on activated T cells in particular, especially, in all of the foregoing regards, those disclosed in U.S. Publication No. 2008/0166352 and PCT Publication No. WO 07/011941, which are incorporated herein by reference in their entireties as to such antibodies and related proteins, including but not limited to antibodies designated therein as follow: 16H (having light chain variable and heavy chain variable sequences sequence identification number:1 and sequence identification number:7 respectively therein); 5D (having light chain variable and heavy chain variable sequences sequence identification number:2 and sequence identification number:9 respectively therein); 2H (having light chain variable and heavy chain variable sequences sequence identification number:3 and sequence identification number:10 respectively therein); 43H (having light chain variable and heavy chain variable sequences sequence identification number:6 and sequence identification number:14 respectively therein); 41H (having light chain variable and heavy chain variable sequences sequence identification number:5 and sequence identification number:13 respectively therein); and 15H (having light chain variable and heavy chain variable sequences sequence identification number:4 and sequence identification number:12 respectively therein), each of which is individually and specifically incorporated by reference herein in its entirety fully as disclosed in the foregoing publication;

IL-15 specific antibodies, peptibodies, and related proteins, and the like, such as, in particular, humanized monoclonal antibodies, particularly antibodies such as those disclosed in U.S. Publication Nos. 2003/0138421; 2003/023586; and 2004/0071702; and U.S. Pat. No. 7,153,507, each of which is incorporated herein by reference in its entirety as to IL-15 specific antibodies and related proteins, including peptibodies, including particularly, for instance, but not limited to, HuMax IL-15 antibodies and related proteins, such as, for instance, 146B7;

IFN gamma specific antibodies, peptibodies, and related proteins and the like, especially human IFN gamma specific antibodies, particularly fully human anti-IFN gamma antibodies, such as, for instance, those described in U.S. Publication No. 2005/0004353, which is incorporated herein by reference in its entirety as to IFN gamma specific antibodies, particularly, for example, the antibodies therein designated 1118; 1118*; 1119; 1121; and 1121*. The entire sequences of the heavy and light chains of each of these antibodies, as well as the sequences of their heavy and light chain variable regions and complementarity determining regions, are each individually and specifically incorporated by reference herein in its entirety fully as disclosed in the foregoing publication and in Thakur et al. (1999), Mol. Immunol. 36:1107-1115. In addition, description of the properties of these antibodies provided in the foregoing publication is also incorporated by reference herein in its entirety. Specific antibodies include those having the heavy chain of sequence identification number:17 and the light chain of sequence identification number:18; those having the heavy chain variable region of sequence identification number:6 and the light chain variable region of sequence identification number: 8; those having the heavy chain of sequence identification number:19 and the light chain of sequence identification number:20; those having the heavy chain variable region of sequence identification number:10 and the light chain variable region of sequence identification number:12; those having the heavy chain of sequence identification number:32 and the light chain of sequence identification number:20; those having the heavy chain variable region of sequence identification number:30 and the light chain variable region of sequence identification number:12; those having the heavy chain sequence of sequence identification number:21 and the light chain sequence of sequence identification number:22; those having the heavy chain variable region of sequence identification number:14 and the light chain variable region of sequence identification number:16; those having the heavy chain of sequence identification number:21 and the light chain of sequence identification number:33; and those having the heavy chain variable region of sequence identification number:14 and the light chain variable region of sequence identification number:31, as disclosed in the foregoing publication. A specific antibody contemplated is antibody 1119 as disclosed in the foregoing U.S. publication and having a complete heavy chain of sequence identification number:17 as disclosed therein and having a complete light chain of sequence identification number:18 as disclosed therein;

TALL-1 specific antibodies, peptibodies, and the related proteins, and the like, and other TALL specific binding proteins, such as those described in U.S. Publication Nos. 2003/0195156 and 2006/0135431, each of which is incorporated herein by reference in its entirety as to TALL-1 binding proteins, particularly the molecules of Tables 4 and 5B, each of which is individually and specifically incorporated by reference herein in its entirety fully as disclosed in the foregoing publications;

Parathyroid hormone (“PTH”) specific antibodies, peptibodies, and related proteins, and the like, such as those described in U.S. Pat. No. 6,756,480, which is incorporated herein by reference in its entirety, particularly in parts pertinent to proteins that bind PTH;

Thrombopoietin receptor (“TPO-R”) specific antibodies, peptibodies, and related proteins, and the like, such as those described in U.S. Pat. No. 6,835,809, which is herein incorporated by reference in its entirety, particularly in parts pertinent to proteins that bind TPO-R;

Hepatocyte growth factor (“HGF”) specific antibodies, peptibodies, and related proteins, and the like, including those that target the HGF/SF:cMet axis (HGF/SF:c-Met), such as the fully human monoclonal antibodies that neutralize hepatocyte growth factor/scatter (HGF/SF) described in U.S. Publication No. 2005/0118643 and PCT Publication No. WO 2005/017107, huL2G7 described in U.S. Pat. No. 7,220,410 and OA-5d5 described in U.S. Pat. Nos. 5,686,292 and 6,468,529 and in PCT Publication No. WO 96/38557, each of which is incorporated herein by reference in its entirety, particularly in parts pertinent to proteins that bind HGF;

TRAIL-R2 specific antibodies, peptibodies, related proteins and the like, such as those described in U.S. Pat. No. 7,521,048, which is herein incorporated by reference in its entirety, particularly in parts pertinent to proteins that bind TRAIL-R2;

Activin A specific antibodies, peptibodies, related proteins, and the like, including but not limited to those described in U.S. Publication No. 2009/0234106, which is herein incorporated by reference in its entirety, particularly in parts pertinent to proteins that bind Activin A;

TGF-beta specific antibodies, peptibodies, related proteins, and the like, including but not limited to those described in U.S. Pat. No. 6,803,453 and U.S. Publication No. 2007/0110747, each of which is herein incorporated by reference in its entirety, particularly in parts pertinent to proteins that bind TGF-beta;

Amyloid-beta protein specific antibodies, peptibodies, related proteins, and the like, including but not limited to those described in PCT Publication No. WO 2006/081171, which is herein incorporated by reference in its entirety, particularly in parts pertinent to proteins that bind amyloid-beta proteins. One antibody contemplated is an antibody having a heavy chain variable region comprising sequence identification number:8 and a light chain variable region having sequence identification number:6 as disclosed in the foregoing publication;

c-Kit specific antibodies, peptibodies, related proteins, and the like, including but not limited to those described in U.S. Publication No. 2007/0253951, which is incorporated herein by reference in its entirety, particularly in parts pertinent to proteins that bind c-Kit and/or other stem cell factor receptors;

OX40L specific antibodies, peptibodies, related proteins, and the like, including but not limited to those described in U.S. Publication No. 2006/0002929, which is incorporated herein by reference in its entirety, particularly in parts pertinent to proteins that bind OX40L and/or other ligands of the OX40 receptor; and

Other exemplary proteins, including Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Neulasta® (pegylated filgastrim, pegylated G-CSF, pegylated hu-Met-G-CSF); Neupogen® (filgrastim, G-CSF, hu-MetG-CSF); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Vectibix® (panitumumab); Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFa mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRa antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); NVS Antibody #1; and NVS Antibody #2.

Also included can be a sclerostin antibody, such as but not limited to romosozumab, blosozumab, or BPS 804 (Novartis). Further included can be therapeutics such as rilotumumab, bixalomer, trebananib, ganitumab, conatumumab, motesanib diphosphate, brodalumab, vidupiprant, panitumumab, denosumab, NPLATE, PROLIA, VECTIBIX or XGEVA.

Additionally, included in the device can be a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab), as well as molecules, variants, analogs or derivatives thereof as disclosed in the following patents or patent applications, each of which is herein incorporated by reference in its entirety for all purposes: U.S. Pat. No. 8,030,547, U.S. Publication No. 2013/0064825, WO2008/057457, WO2008/057458, WO2008/057459, WO2008/063382, WO2008/133647, WO2009/100297, WO2009/100318, WO2011/037791, WO2011/053759, WO2011/053783, WO2008/125623, WO2011/072263, WO2009/055783, WO2012/0544438, WO2010/029513, WO2011/111007, WO2010/077854, WO2012/088313, WO2012/101251, WO2012/101252, WO2012/101253, WO2012/109530, and WO2001/031007.

Also included can be talimogene laherparepvec or another oncolytic HSV for the treatment of melanoma or other cancers. Examples of oncolytic HSV include, but are not limited to talimogene laherparepvec (U.S. Pat. Nos. 7,223,593 and 7,537,924); OncoVEXGALV/CD (U.S. Pat. No. 7,981,669); OrienX010 (Lei et al. (2013), World J. Gastroenterol., 19:5138-5143); G207, 1716; NV1020; NV12023; NV1034 and NV1042 (Vargehes et al. (2002), Cancer Gene Ther., 9(12):967-978).

Also included are TIMPs. TIMPs are endogenous tissue inhibitors of metalloproteinases (TIMPs) and are important in many natural processes. TIMP-3 is expressed by various cells or and is present in the extracellular matrix; it inhibits all the major cartilage-degrading metalloproteases, and may play a role in role in many degradative diseases of connective tissue, including rheumatoid arthritis and osteoarthritis, as well as in cancer and cardiovascular conditions. The amino acid sequence of TIMP-3, and the nucleic acid sequence of a DNA that encodes TIMP-3, are disclosed in U.S. Pat. No. 6,562,596, issued May 13, 2003, the disclosure of which is incorporated by reference herein. Description of TI MP mutations can be found in U.S. Publication No. 2014/0274874 and PCT Publication No. WO 2014/152012.

Also included are antagonistic antibodies for human calcitonin gene-related peptide (CGRP) receptor and bispecific antibody molecule that target the CGRP receptor and other headache targets. Further information concerning these molecules can be found in PCT Application No. WO 2010/075238.

Additionally, bispecific T cell engager (BITE®) antibodies, e.g. BLINCYTO® (blinatumomab), can be used in the device. Alternatively, included can be an APJ large molecule agonist e.g., apelin or analogues thereof in the device. Information relating to such molecules can be found in PCT Publication No. WO 2014/099984.

In certain embodiments, the medicament comprises a therapeutically effective amount of an anti-thymic stromal lymphopoietin (TSLP) or TSLP receptor antibody. Examples of anti-TSLP antibodies that may be used in such embodiments include, but are not limited to, those described in U.S. Pat. Nos. 7,982,016, and 8,232,372, and U.S. Publication No. 2009/0186022. Examples of anti-TSLP receptor antibodies include, but are not limited to, those described in U.S. Pat. No. 8,101,182. In particularly preferred embodiments, the medicament comprises a therapeutically effective amount of the anti-TSLP antibody designated as A5 within U.S. Pat. No. 7,982,016.

Although the drug delivery devices, methods, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention. For example, components described herein with reference to certain kinds of drug delivery devices, such as on-body injector drug delivery devices or other kinds of drug delivery devices, can also be utilized in other kinds of drug delivery devices, such as autoinjector drug delivery devices.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method of assembling a platform drug delivery device, the method comprising:
providing a set of base components for the drug delivery device;
identifying, based on at least one desired characteristic of the drug delivery device, a rear sub-assembly for the drug delivery device from a group of rear sub-assemblies;
selecting the identified rear sub-assembly;
identifying, based on at least one desired characteristic of the drug delivery device, a front sub-assembly for the drug delivery device from a group of front sub-assemblies;
selecting the identified front sub-assembly; and
assembling the drug delivery device using the set of base components, the rear sub-assembly, and the front sub-assembly.

2. The method of claim 1, wherein the at least one desired characteristic comprises at least one of a drug viscosity or a drug volume.

3. The method of claim 1, wherein each rear sub-assembly in the group of rear sub-assemblies includes at least one of (a) a different drive mechanism, and (b) a different syringe assembly.

4. (canceled)

5. The method of claim 3, wherein each syringe assembly includes a syringe that is constructed from one of glass or a polymeric material.

6. (canceled)

7. The method of claim 1, further comprising applying a skin to the drug delivery device.

8. The method of claim 7, wherein the skin is selected based on at least one attribute from an intended user group.

9. A method of assembling a platform drug delivery device, the method comprising:
providing a set of base components for the drug delivery device;
identifying, based on at least one desired characteristic of the drug delivery device, a first sub-assembly for the drug delivery device from a first group of selectable sub-assemblies;
selecting the identified first sub-assembly;
identifying, based on the at least one desired characteristic, a second sub-assembly for the drug delivery device from a second group of selectable sub-assemblies;
selecting the identified second sub-assembly;
identifying, based on the at least one desired characteristic, a third sub-assembly for the drug delivery device from a third group of selectable sub-assemblies;
selecting the identified third sub-assembly;
assembling the drug delivery device using the set of base components, the first sub-assembly, the second sub-assembly, and the third sub-assembly.

10. The method of claim 9, wherein the at least one characteristic comprises at least one of a drug viscosity or a drug volume.

11. The method of claim 9, wherein the first group of selectable sub-assemblies comprises a plurality of pre-filled syringe assemblies.

12. The method of claim 9, wherein the second group of selectable sub-assemblies comprises a plurality of drive assemblies.

13. The method of claim 12, wherein the plurality of drive assemblies include a plurality of torque springs, each of the plurality of torque springs having distinct characteristics.

14. The method of claim 9, wherein the third group of selectable sub-assemblies comprises a plurality of volume adapters.

15. The method of claim 9, further comprising applying a skin to the drug delivery device.

16. The method of claim 15, wherein the skin is selected based on at least one attribute from an intended user group.

17-21. (canceled)

22. A platform system for assembling a drug delivery device, the system comprising:
a set of base components for the drug delivery device;
a first group of selectable sub-assemblies for the drug delivery device;
a second group of selectable sub-assemblies for the drug delivery device; and
a third group of selectable sub-assemblies for the drug delivery device;
wherein the drug delivery device is assembled by using at least one desired characteristic of the drug delivery device to identify and select a first sub-assembly from the first group of selectable sub-assemblies, a second sub-assembly from the second group of selectable sub-assemblies, and a third sub-assembly from the third group of selectable sub-assemblies and coupling the set of base components to the first group of selectable sub-assemblies, the second group of selectable sub-assemblies, and the third group of selectable sub-assemblies.

23. The platform system of claim 22, wherein the at least one characteristic comprises at least one of a drug viscosity or a drug volume.

24. The platform system of claim 22, wherein the first group of selectable sub-assemblies comprises a plurality of pre-filled syringe assemblies.

25. The platform system of claim 22, wherein the second group of selectable sub-assemblies comprises a plurality of drive assemblies.

26. The platform system of claim 25, wherein the plurality of drive assemblies include a plurality of torque springs, each of the plurality of torque springs having distinct characteristics.

27. The platform system of claim 22, wherein the third group of selectable sub-assemblies comprises a plurality of volume adapters.
